(19) United States
(12) Patent Application Publication
(10) Pub. No.: US 2026/0263649 A1
(43) Pub. Date: Sep. 10, 2026

(54) DECONTAMINATION METHOD, IN PARTICULAR FOR PHARMACEUTICAL SYSTEMS AND APPLICATIONS, AND DECONTAMINATION ARRANGEMENT FOR PHARMACEUTICAL APPLICATIONS

(71) Applicant: OPTIMA PHARMA CONTAINMENT GMBH, Radolfzell (DE)

(72) Inventors: Stefan Knellwolf, Goldach (CH); Thomas Kassner, Radolfzell (DE); Andreas Demmler, Konstanz (DE); Lydia Bernhard, Stockach (DE)

(73) Assignee: OPTIMA PHARMA CONTAINMENT GMBH, Radolfzell (DE)

(21) Appl. No.: 19/469,182

(22) PCT Filed: Mar. 26, 2024
(86) PCT No.: PCT/EP2024/058134

§ 371 Date: Sep. 25, 2025

(30) Foreign Application Priority Data
Mar. 31, 2023 (EP) 23166182.8

(51) Int. Cl. A61L 2/186; A61L 2/22; A61L 103/10; A61L 103/20

(52) U.S. Cl. CPC A61L 2/186; A61L 2/22; A61L 2103/10; A61L 2103/20; A61L 2202/122; A61L 2202/15

(57) ABSTRACT

The invention relates to a decontamination method, in particular for applications in pharmaceutical engineering, including the steps of: introducing a decontamination agent into at least one gas line (07) serving to supply at least one atomizer (04) with a carrier gas stream, preferably compressed air, said atomizer being disposed in an isolator space (02) and being preferably realized as two-fluid nozzle, the at least one atomizer (04) being supplied with decontamination liquid, preferably hydrogen peroxide, from a decontamination agent storage container (06) via valve means controllable by means of electronic control means in order to generate a decontamination aerosol in a decontamination operation of the isolator space (02), the at least one atomizer (04) being connected to a gas pressure source (08) in a gas-conducting manner via the gas line (07) and a filter (11) for filtering the carrier gas stream being disposed in the gas line (07) between the gas pressure source (08) and the atomizers (04), preferably individually for each atomizer (04).

### BACKGROUND OF THE INVENTION

The present invention relates to a decontamination method, in particular for applications and plants in pharmaceutical engineering. Furthermore, the present invention relates to a decontamination assembly, in particular for applications in pharmaceutical engineering.

In the state of the art, different decontamination assemblies and devices are known to decontaminate an isolator space, which preferably has a plenum space and a manipulator space connected in a gas-conducting manner to the plenum space. Devices having a corresponding isolator space, which is for example realized as an isolator, are used, but by no means exclusively, when filling, packaging, repackaging and treating pharmaceuticals and/or medical engineering products. The decontamination serves the purpose of establishing a germ-free and/or sterile environment in order to prevent a contamination of the substances and products disposed in the isolator space during the operation of said isolator space.

Different approaches for decontaminating the isolator space are known from the state of the art. For example, EP 2 719 962 A1 teaches a hydrogen peroxide evaporator. U.S. Pat. No. 6,010,400 A also provides a hydrogen peroxide evaporator.

However, different approaches are also known from the state of the art, said different approaches producing a decontamination aerosol from a decontamination liquid and a carrier gas or propellant gas, for example, on the basis of atomizers, for example two-fluid nozzles, and distributing it in the isolator space, preferably in the plenum space and manipulator space. Liquid hydrogen peroxide solutions can preferably also be used as a decontamination agent. Such an approach is pursued, for example, in EP 3 409 300 A1, in this case, the decontamination agent, in particular the decontamination liquid, being provided via a ring line connected to the atomizers, in addition to the use of atomizers, in particular two-fluid nozzles.

Basically, the production of the decontamination aerosol from decontamination agents, in particular decontamination liquid and carrier gas, for example compressed air, is to serve to prevent or remove possible decontaminations in the isolator space.

However, the risk also exists that decontaminations also or in spite of corresponding filter mechanisms enter the isolator space via the carrier gas or the carrier gas stream. The gas lines and filters of the gas lines for the carrier gas stream themselves are also potential sources for impurities and contaminations, which can then correspondingly be introduced into the isolator space by means of the carrier gas stream. In this case, it is particularly critical that, in certain situations of decontamination of the isolator space, the atomizers, in particular dual-fluid nozzles, continue to eject carrier gas stream or supply it into the isolator space even if no more decontamination agent or no more decontamination liquid is being atomized or provided. This continued flow of the carrier gas stream, for example from a gas pressure source to the atomizers and thus through the corresponding gas lines and/or filters, serves, for example, to test the flow rate of the carrier gas stream and to test the tightness of the system.

Particularly in such situations in which the carrier gas stream continues to be introduced from the gas pressure source into the isolator space via the gas line and/or filter without decontamination agents, in particular decontamination fluid, being dispensed via the atomizers, the risk exists that impurities or contaminations enter the isolator space and, from there, enter a product or agent which is handled, for example filled, packaged, repackaged, or dosed, in the isolator space.

### SUMMARY OF THE INVENTION

In the light of the above, the object of the present invention consists in further developing known decontamination assemblies and decontamination methods, in particular for applications in pharmaceutical engineering, for decontaminating an isolator space in such a manner that the disadvantages in the state of the art are overcome and, in particular, excluded that decontaminations are introduced into the isolator space via the carrier gas stream conveyed through a gas line and serving for the supply of the atomizers and for the production of a decontamination aerosol.

The aforementioned object is attained with the features of a decontamination method as disclosed herein. Referring to a decontamination assembly, the abovementioned object is also attained with the features disclosed herein.

Advantageous embodiments of the invention are the subject matter of the subsequent description, the description of the figures, the figures and the claims.

Basically, features disclosed below in connection with the method shall also be considered correspondingly disclosed and claimable in connection with the method, and vice versa.

With regard to the decontamination method, in particular for applications in pharmaceutical engineering, as defined by the present invention, the intention is that a decontamination agent is introduced into at least one gas line serving to supply at least one atomizer with a carrier gas stream, preferably compressed air, said atomizer being disposed in an isolator space and being preferably realized as two-fluid nozzle, the at least one atomizer being supplied with decontamination agent, preferably hydrogen peroxide, from a decontamination agent storage container via valve means controllable by means of electronic control means in order to generate a decontamination aerosol in a decontamination operation of the isolator space, and, furthermore, the at least one atomizer being connected to a gas pressure source in a gas-conducting manner via the gas line and a filter for filtering the carrier gas stream being disposed in the gas line between the gas pressure source and the atomizer, preferably individually for each atomizer.

The method according to the invention is thus based on the basic idea of also introducing decontamination agent, at least in certain decontamination intervals, into the gas line, which otherwise is used only to conduct the carrier gas stream in order to supply the atomizer, preferably the two-fluid nozzle, with the carrier gas or propellant gas in order to generate or atomize a decontamination aerosol from a decontamination agent, preferably a decontamination liquid.

As will be further explained in detail below, this allows the gas line and/or the filters within the gas line to be correspondingly decontaminated such that a subsequent carrier gas stream, for example compressed air, cannot be impurified or contaminated by contaminations being present within the gas line and/or filters.

In other words, the gas line for providing the carrier gas stream of the atomizers is diverted from its intended use for a short period of time and used or employed for guiding decontamination agent in order to decontaminate the gas line itself or the inside of the gas line and/or filter or parts of filters within the gas line. The decontamination of the gas line(s) can advantageously be performed before and/or after the decontamination of the isolator space. Particularly advantageously, in particular before the decontamination of the gas lines, filters of the gas lines can also be exchanged at the same time.

With respect to the basic decontamination method for decontaminating the isolator space, reference can generally be made to EP 3 409 300 A1, the content of which with respect to the design of the decontamination assembly and the course of the decontamination method is incorporated into the present disclosure by reference.

However, the application of the decontamination method according to the invention and the decontamination assembly according to the invention is not limited to the devices and methods disclosed in the abovementioned description. Basically, the method according to the invention and the device according to the invention can be used in every decontamination method and in every decontamination assembly in which a carrier gas stream is generated by a gas pressure source and is conducted to at least one atomizer via a gas line in order to atomize a decontamination agent, for example liquid hydrogen peroxide, in the area of the atomizers or upon exiting the atomizer and convert it into a decontamination aerosol.

In a first advantageous embodiment of the method, it can be advantageous that the liquid decontamination agent, for example liquid H₂O₂, is introduced into the gas line and is delivered with the carrier gas stream into the isolator space via the atomizer. This configuration has several advantages. On the one hand, the carrier gas stream, which is in the gas line anyway, can be used in order to deliver the introduced, liquid decontamination agent. Accordingly, no additional conveying mechanisms or other arrangements have to be provided in order to deliver the liquid decontamination agent introduced into the gas line. The use of a liquid decontamination agent, for example liquid hydrogen peroxide, has the advantage that the surfaces to be decontaminated, in particular the gas line, can be wetted particularly well and effectively using said liquid decontamination agent and an effective decontamination of the gas line can thus be carried out.

In the scope of the present disclosure, mention is made in part of an atomizer and the associated gas line for providing the carrier gas stream. However, this should by no means be understood as limiting the invention to exactly one atomizer. Instead, in decontamination methods and decontamination assemblies of this kind, often, a plurality of atomizers is provided which are disposed at different locations of the isolator space, preferably the plenum space and/or manipulator space, in order to produce or build up a corresponding concentration of decontamination aerosol in the spaces as effectively and evenly as possible. In principle, it can however be advantageous if the introduction of the decontamination agent, for example of the liquid decontamination agent, according to the embodiment described above is not carried out at a central location, for example directly behind the gas pressure source, but is carried out downstream with respect to the gas pressure source (in relation to the flow direction of the carrier gas stream during the atomizing of the decontamination agent by the atomizers) such that preferably each individual gas line, which is connected to an atomizer in a gas-conducting manner, or every branch of a corresponding gas line system can be decontaminated individually and separately by the introduction of the decontamination agent.

In another, advantageous configuration the liquid decontamination agent may be introduced at a discharge point in the gas line, said discharge point preferably being individual for each atomizer. Using a discharge point, preferably between the gas pressure source and the atomizer, ensures that the decontamination agent, in particular the liquid decontamination agent, flows through or passes through the necessary and relevant areas of the gas line, for example starting from a corresponding filter. As already explained above, the individual discharge or the individual introduction of liquid decontamination agent at an individual discharge point, which is assigned to an atomizer, in the gas line can ensure that to each individual gas line individually supplying an atomizer with a carrier gas stream, sufficient liquid decontamination agent is applied and the gas line is consequently safely decontaminated.

In another, particularly advantageous configuration of the decontamination method, the intention is that the liquid decontamination agent is introduced at a discharge point which is disposed in the gas line between a filter and the atomizer.

This can ensure that, starting from the filter and including a side or a surface of the filter up to the atomizer and the entire gas line lying between them, is purified and decontaminated by the liquid decontamination agent, preferably the liquid hydrogen peroxide. In this process, the quantity or the volume of liquid decontamination agent can possibly advantageously be reduced if the section of the gas line between the filter and the atomizer is not too large, preferably is in the range of a few meters. Preferably, the discharge point is disposed as close as possible to a position of the filter along the gas line.

In another, also particularly advantageous embodiment of the method, for the decontamination of the gas line, a container having a, preferably pre-dosed, volume of liquid decontamination agent is coupled to a closeable coupling of the gas line at the discharge point during a temporary connection and is emptied during the temporary connection. For example, prefabricated or pre-filled cartridges, cannulas, or syringes can serve as containers, which are filled with a corresponding decontamination agent, for example liquid hydrogen peroxide, and are then coupled to the discharge point via a closable coupling of the gas line, and the transfer of the liquid decontamination agent into the gas line takes place during the connection between the container and the coupling. After the introduction of the liquid decontamination agent into the gas line has been completed, the container can again be separated from the discharge point via the closable coupling and the coupling can be closed. Preferably, before and/or after establishing the connection between the closable coupling and the container, a part of the closable coupling or the entire closable coupling is first decontaminated and/or disinfected to prevent that a contamination of the gas line results via the coupling.

In another, particularly advantageous configuration of the method, for decontaminating the gas line, the liquid decontamination agent may be provided via a permanent feed line conveying decontamination agent from the decontamination agent storage container into the gas line, preferably by means of corresponding conveying means via an at least indirect connection between the decontamination liquid container and the discharge point. It is true that this configuration causes a somewhat greater plant-specific complexity due to the permanent lines or connections between the decontamination liquid container and the discharge points on the part of the gas line to be decontaminated, at the same time, however, this design enables a largely fully automatic decontamination of the gas line using the decontamination agent storage container provided anyway for the decontamination method of the isolator space in a particularly advantageous and synergetic manner. In other words, this means that a second connection between the decontamination agent storage container and the atomizer is established in the present embodiment in the decontamination method for decontaminating the gas line, the first connection providing, for example, the feeding of the atomizer with decontamination agent in the decontamination method of the isolator space, in which a carrier gas stream, for example compressed air, is provided via the gas line. The second connection between the decontamination agent storage container and the atomizer runs to the discharge point of the gas line via the at least indirect connection, from there through the gas line and also to the atomizer, the preferred conveying means then conveying decontamination agent, preferably liquid decontamination agent, up to the discharge point, from where it is advantageously delivered by means of a carrier gas stream, preferably delivered by the atomizer, said carrier gas stream also otherwise being conveyable through the gas line.

In another, advantageous embodiment of the method, the decontamination agent may be introduced into the gas line in the form of a decontamination aerosol. In the technology described at the beginning, the use of decontamination aerosol for decontaminating the isolator space has already proved to be very successful. Similarly, as described at the beginning, also corresponding agents are provided and corresponding methods established in plants and decontamination methods to generate decontamination aerosol and stabilize their concentration. Therefore, by synergetically exploiting these methods and device features, the decontamination of the gas line ending in the atomizer can also be effected in a particularly advantageous manner via corresponding decontamination aerosols.

In addition, in an advantageous further development, the decontamination aerosol may be introduced from the isolator space into the gas line, preferably by passing backwards through the atomizer, and introduced, in particular sucked in, into the gas line by means of a carrier gas stream having a reverse flow direction to the carrier gas stream during the decontamination operation of the isolator space. In other words, the present embodiment intends in a particularly advantageous manner that decontamination aerosol in the isolator space, as it is also generated in the case of the decontamination of the isolator itself, is guided, in particular sucked, back from the isolator space into the gas line via the respective openings of the atomizers by reversely connecting or reversely controlling the gas pressure source of the atomizers, which are connected to the gas lines. For this purpose, merely a corresponding concentration of decontamination aerosol has to be generated and maintained in the isolator space and the gas pressure source has to be operated in such a manner that no positive pressure, which causes a carrier gas stream from the gas pressure source toward the atomizers, occurs in the gas lines, but that, conversely, a negative pressure, which ensures that a carrier gas stream flows from the isolator space via the atomizers and the gas line—and, if necessary, through possible filters in the gas line—toward the gas pressure source, is generated in the gas lines.

For example, if the gas pressure source is realized as a switchable pump, the reversing of the carrier gas stream can be carried out easily and effectively. In this case, the gaseous parts of the decontamination agent of the decontamination aerosol can also pass through filters, in particular hydrophobic filters, in the gas line and achieve a decontamination of the gas line beyond these filters. Liquid components of the decontamination aerosol can accumulate and/or condense on the inner surfaces of the gas line in a desired and intended manner in order to decontaminate the inner surface of the gas line there. It is particularly advantageous to ensure that the relative negative pressure generated by the gas pressure source does not turn out to be too great, as otherwise the droplets or liquid particles of the decontamination aerosol will transition too quickly or even abruptly into the gas phase in the form of flash evaporation within the gas line, thereby limiting or restricting the decontamination effect in the gas line. The negative pressure in the gas line is advantageously between 500 and 950 mbar.

The introduced, in particular sucked in, decontamination aerosol can advantageously contain a concentration of the decontamination agent of 400 to 1200 ppm. If the concentration of decontamination agent in the decontamination aerosol is too low, a reliable decontamination in the gas line can no longer be guaranteed.

In another, particularly advantageous configuration of the decontamination method, a first part of an entirety of several atomizers may atomize a decontamination aerosol in the isolator space while a second part of the entirety of the several atomizers may take the decontamination aerosol from the isolator space and introduce it, in particular suck it in, in particular, into the gas line (EM) of the second part of the entirety of the atomizers. It has been found that a decontamination aerosol, which is initially produced via a part or the entirety of the several atomizers, in the isolator space is not sufficient to decontaminate all gas lines simultaneously or one after the other, preferably by correspondingly sucking in the decontamination aerosol from the isolator space via the atomizers.

However, it has been successfully achieved that a decontamination aerosol consisting of decontamination agent and a carrier gas stream is delivered in addition by a first part or a first group of atomizers in order to maintain a minimum concentration of decontamination agent or decontamination aerosol in at least one area of the isolator space, in order to simultaneously take, in particular suck in, a part of the decontamination aerosol via a second part of the entirety of the several atomizers and use it to decontaminate the adjoining or adjacent gas lines.

Preferably the method is performed in such a manner that the atomizers producing or supplying decontamination aerosol and the atomizers drawing off or sucking in decontamination aerosols are disposed in spatial proximity to each other. Furthermore, advantageously, the corresponding decontamination of gas lines may be carried out successively or individually one after the other. Alternatively, a decontamination of the gas lines in groups can also be provided. The respective identification of appropriate sequences and/or appropriate groups of atomizers also depends on the geometry and exact structure of the isolator space and the arrangement of the atomizers there, for example, in a plenum space and a treatment space.

In this configuration, it can be particularly advantageous if several gas pressure sources are provided and can be operated independently of each other or if corresponding negative pressures and positive pressures can be generated and maintained simultaneously with one gas pressure source, for example via a variable/switchable gas-conducting connection to an inlet side and an outlet side of a pump.

In another, particularly advantageous embodiment of the method, the entirety of the several atomizers may atomize a decontamination aerosol into the isolator space before introducing, in particular sucking in, the decontamination aerosol into the gas line. This allows an initial concentration to be achieved quickly and effectively before atomizers and connected gas lines begin then to introduce, in particular suck in, decontamination aerosol individually or in groups, while the other part of the entirety of the atomizers delivers in addition decontamination aerosol and, thus, prevents the concentration of decontamination agent or decontamination aerosol in the isolation space from falling too strongly or too quickly.

In another, particularly advantageous embodiment of the method, the flow direction of the carrier gas stream may be reversed after the introduction, in particular the sucking in, of the decontamination aerosol into the gas line and, preferably liquid, decontamination agent and/or decontamination aerosol remaining in the gas line may be delivered via the atomizers into the isolator space by means of the carrier gas stream. This prevents residues of the decontamination agent and/or decontamination aerosol from remaining in the gas lines and forming deposits and/or contaminations there. As already explained above, for the embodiment which intends sucking in decontamination aerosol via the atomizers into the gas lines, it is in any case necessary that the direction of the carrier gas stream through the gas line can be reversed. Accordingly, in a corresponding further development, the direction of the carrier gas stream may particularly advantageously be reversed again after the decontamination of the gas lines has been completed in order to expel residues of the decontamination agent and/or the decontamination aerosol again from the gas lines into the isolator space via the atomizers. This also makes particular sense because corresponding agents and methods, which allow and facilitate a decomposition and a degradation of decontamination agent and/or decontamination aerosol located in the isolator space, are known from the general decontamination of the isolator space and typically provided.

The abovementioned object is also attained by a decontamination assembly, in particular for applications in pharmaceutical engineering, said decontamination assembly comprising at least one isolator space and a decontamination agent storage container from which at least one, preferably several, atomizers, which are disposed in the isolator space or are disposed on a surface delimiting the isolator space and which are preferably realized as two-fluid nozzles, are suppliable with decontamination liquid, preferably hydrogen peroxide, via valve means controllable by means of electronic control means in order to generate a decontamination aerosol, the atomizers being connected to a gas pressure source, in particular a source of compressed air, in a gas-conducting manner via a gas line in order to generate a carrier gas stream for a decontamination aerosol to be generated and a filter for filtering the carrier gas stream being disposed in the gas line between the gas pressure source and the atomizers, preferably individually for each atomizer. According to the invention, furthermore, the intention is that the distance between the filter and the atomizer in a gas line is less than 5 m and/or that the gas-conducting connection comprises a discharge point between the gas pressure source and the atomizers, said discharge point being preferably provided individually for each atomizer and/or filter and serving for the discharge of decontamination liquid.

With such a decontamination assembly, it is advantageously achieved that the decontamination methods described above can be implemented or performed. Liquid decontamination agent can be introduced into the gas line either manually, by hand, or in a partially automated/fully automated manner through the discharge point in the gas line.

When decontamination aerosol is sucked into the gas line via the atomizers, the section of the gas line which can be decontaminated is limited starting from the atomizers. In other words, the decontamination reliability decreases with increasing distance from the atomizer when the decontamination aerosol is sucked into the gas line. Also, when introducing, in particular injecting, liquid decontamination agent through a discharge point of the gas line, a restricted distance of less than 5 m between the atomizer and a filter can particularly advantageously improve the decontamination of the gas line beyond the filter or between the filter and the atomizer. Therefore, the intention of the invention is or the intention of the invention may be that a corresponding filter, which is disposed at a distance of less than 5 m with respect to the respective atomizer, is provided additionally or alternatively to the discharge point.

In a first advantageous configuration of the decontamination assembly, at least one individual container for liquid decontamination agent may be separably connected to a closeable coupling provided at the discharge point of the gas line. In this case, the individual container can also be used successively or consecutively to be coupled to or docked with different gas lines at corresponding closeable couplings in order to provide or introduce a decontamination liquid or liquid decontamination agent in each case. Preferably, the closeable coupling can be designed in such a manner that a separable connection to the container is possible and, at the same time, simple decontamination of the closeable coupling itself, in particular in the area of the connection to the container, is enabled. Also, advantageously, the container may be designed in such a manner that it can be decontaminated, for example decontaminated by hand, in the area of the connection to the closeable coupling.

In another, advantageous embodiment of the decontamination assembly, the discharge point may be connected to the decontamination agent storage container via an indirect connection. The connection may, for example, be realized as an additional hose line. Furthermore, a correspondingly branched system of hose lines can be provided in order to introduce liquid decontamination agent from the decontamination agent storage container to the discharge point of the respective gas line and, from there, into the gas line. By means of these at least indirect connections, in particular lines, a partially automated or fully automated introduction of the liquid decontamination agent into the gas line can be achieved.

In another, advantageous configuration of the decontamination assembly, furthermore, the decontamination assembly may comprise a negative pressure source which is connected in a fluid-conducting manner to a gas line or a part of a gas line. A carrier gas stream can be provided at the atomizer via the gas line, preferable starting from a gas pressure source.

Preferably, a fluid-conducting connection of the negative pressure source, preferably a negative pressure pump or vacuum pump, is switchable with the fluid line. This can be advantageously realized by means of corresponding valves. In a first valve or switching position, the access or the connection between the gas line and the fluid line to the negative pressure source is closed and, at the same time, the fluid line between a gas pressure source and an atomizer is opened. In this state, the carrier gas stream can be dispensed at the atomizer and atomize decontamination agent there. At the same time, the carrier gas stream cannot be sucked by the negative pressure source or sucked out of the gas line. In a second valve or switching position, the fluid line between the gas pressure source and the atomizer is interrupted and, instead, the fluid line between the negative pressure source and the atomizer is opened. The carrier gas stream at the switching point or valve cannot reach the atomizer. Instead, a negative pressure can be produced in a part of the gas line, said negative pressure ensuring at the atomizer that gas or aerosol is sucked from the isolator space.

Thus, the introduction of a decontamination aerosol from the isolator space into the gas line via the atomizers is achieved and made possible in a particularly advantageous manner. Due to the switchable connection of the atomizers to the gas pressure source and the negative pressure source, advantageously, an individual switching or switching in groups of individual gas lines and atomizers can take place in such a manner that a part of the atomizers continues to dispense or atomize a decontamination agent, while other atomizers suck in the aerosol given off into the isolator space.

Advantageously, a filter can be disposed between the switchable connection of the gas line to the gas pressure source and the negative pressure source and the atomizer. This ensures that the gas line is decontaminated from the atomizer up to at least the filter when sucking in decontamination aerosol from the isolator space.

Below, advantageous configurations of the present invention are explained with reference to the exemplary, merely schematic drawings.

### BRIEF DESCRIPTION OF THE DRAWINGS

In the figures:

FIG. 1a: shows a schematic illustration of a detail of a decontamination assembly according to the invention in a first embodiment;

FIG. 1b: shows a schematic illustration of a detail of a decontamination assembly according to the invention in a second embodiment;

FIG. 2: shows a schematic detail from a decontamination assembly according to the invention in a second embodiment;

FIG. 3: shows a flow chart for a decontamination method according to the invention in a first embodiment;

FIG. 4: shows a flow chart for a decontamination method according to the invention according to a second embodiment;

FIG. 5: shows a flow chart for a decontamination method according to the invention according to a third embodiment.

### DETAILED DESCRIPTION

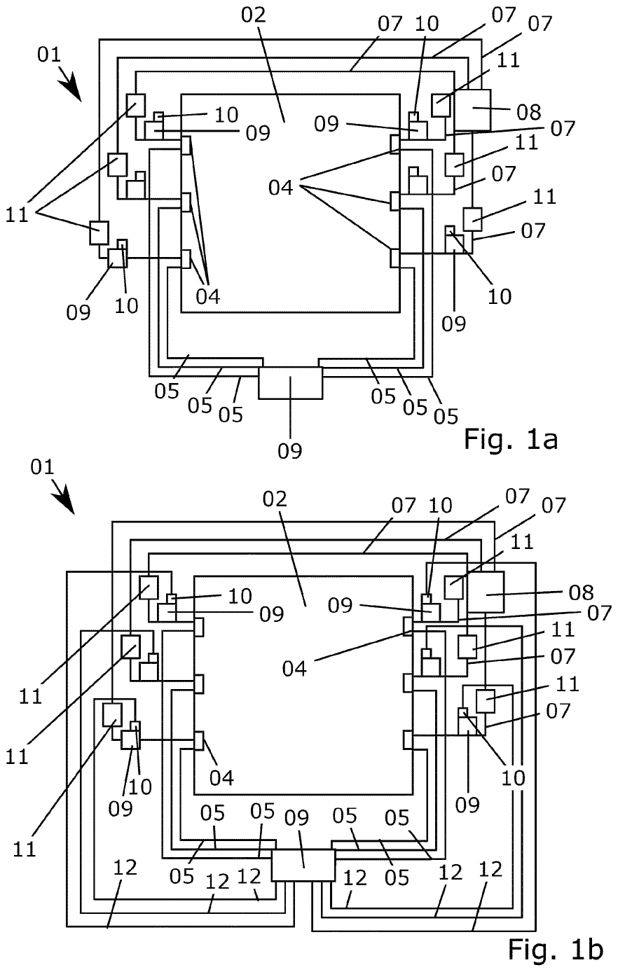

FIG. 1 shows a detail of a decontamination assembly 01. A part of an isolator space 02 is illustrated, at the boundaries of which, in particular in the walls 03 of which, which have corresponding openings to isolator space 02, a plurality of atomizers 04 is disposed. Atomizers 04 are realized, for example, as two-fluid nozzles and are accordingly on the one hand connected with a decontamination agent line 05 to a decontamination agent storage container 06. Moreover, atomizers 04 are connected to a gas pressure source 08 via gas lines 07. Gas pressure source 08 serves to provide a carrier gas stream through gas lines 07 at atomizers 04 in the decontamination method of isolator space 02, said carrier gas stream atomizing a decontamination agent, for example hydrogen peroxide (H₂O₂), brought out or delivered via atomizers 04 and converts it into a decontamination aerosol.

In the embodiment of FIG. 1, discharge points 09 are provided in gas lines 07. Closeable couplings 10 can be realized at discharge points 09. Liquid decontamination agent can be introduced into gas lines 07 via closeable couplings 10 via a detachable connection to a container, in particular a decontamination agent container, wherein said liquid decontamination agent can then be conveyed via a carrier gas stream, which is generated and conveyed by means of gas pressure source 08, through gas lines 07 and from discharge points 09 to atomizers 04 and, from there, into isolator space 02. Thereby, gas line 07 can be decontaminated starting from discharge point 09. In order to prevent an effect of a decontamination of the carrier gas stream or an effect of a decontamination of gas line 07 between discharge points 09 and gas pressure source 08, filters 11 are provided between discharge points 09 and gas pressure source 08, if possible directly adjoining or adjacent to discharge points 09, said filters 11 filtering the carrier gas stream supplied by gas pressure source 08.

In the varied illustration of FIG. 1b, the basic structure of decontamination assembly 01 is similar to that in FIG. 1a. Identical reference numerals in FIG. 1b indicate components having the same function. In FIG. 1b, it can be seen that, instead of closeable couplings 10, another, at least indirect, connection 12 is realized between discharge points 09 of gas lines 07 and decontamination agent storage container 06. With said other, at least indirect, connection 12, liquid decontamination agent can be directly taken from decontamination agent storage container 06, for example, via conveying means (not illustrated in detail), for example pumps, and be introduced into gas line 07 at discharge point 06 or discharge points 06. Also with this configuration, it can be particularly advantageous that the introduced, liquid decontamination agent is given off or delivered into isolator space 02 via a corresponding carrier gas stream, as already described with respect to FIG. 1a, from gas lines 07 via atomizers 04.

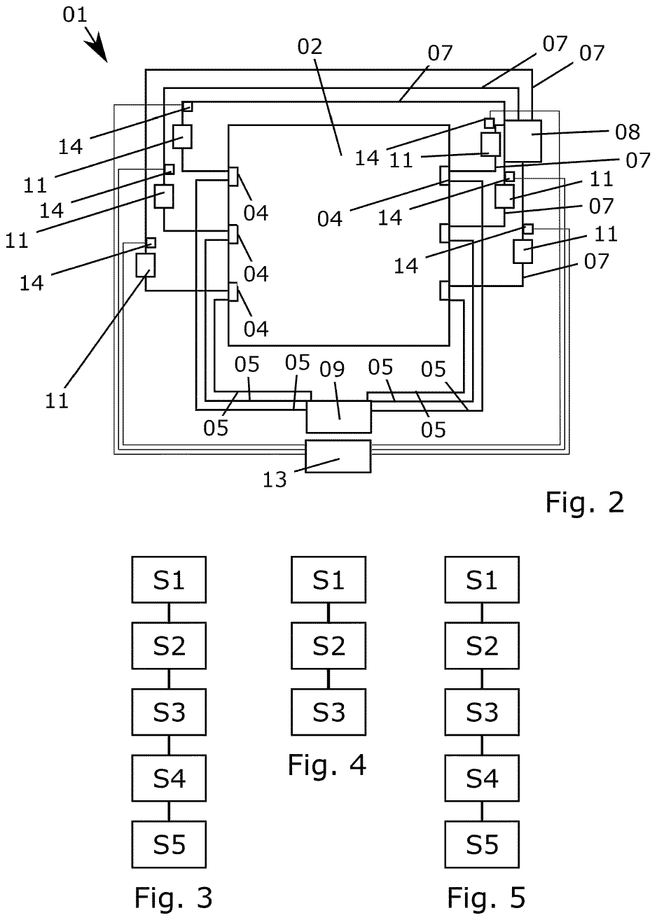

In the configuration of FIG. 2, discharge points 09, closeable couplings 10 and additional, indirect connection 12 having decontamination agent storage container 06 can be dispensed with. Instead, in this embodiment, a switchable connection, preferably a valve 14, is disposed in the gas line, said switchable connection enabling a switchable fluid-conducting connection of atomizer 04 with gas pressure source 08 or negative pressure source 13. Thus, either the carrier gas stream of gas pressure source 08 or a negative pressure of negative pressure source 13 can be applied to gas line 07 in the area between atomizers 04 and valve 14.

The positive pressure or the negative pressure, and, thus, also the flow rate, can be influenced by adjusting valve 14 correspondingly. As will still be described in detail below, in the embodiment of FIG. 2, a decontamination aerosol is produced in isolator space 02, said decontamination aerosol then also being introduced, in particular sucked in, into gas line 07 by a corresponding pressurization of gas lines 07, preferably of a part or a group of gas lines 07, with negative pressure and an accompanying generation of a return gas stream from isolator space 02 via atomizers 04 through gas lines 07 in the direction of negative pressure source 13. The decontamination aerosol which has been sucked in can be used to decontaminate gas lines 07, at least up to filters 11, provided that the concentration of decontamination agent in isolator space 02 is sufficiently high and remains sufficiently high and that the negative pressure in gas lines 07 is not too great. Preferably, a carrier gas stream is dispensed and a decontamination agent atomized via a part of atomizers 04 and connected gas lines 07, while decontamination aerosol from isolator space 02 is sucked in via an atomizer or a group of atomizers.

In the flow chart of FIG. 3, a method is illustrated, which is preferably performed using a decontamination assembly according to FIG. 1a. In a first method step S1, first, filters 11 may be exchanged and replaced with new, sterile or pre-decontaminated filters 11. In a subsequent method step S3, advantageously, corresponding containers having liquid decontamination agent may then be temporarily coupled individually or simultaneously to individual or all closeable couplings 10. In a preceding method step S2, advantageously, closable couplings 10 and/or the containers may first themselves be purified and/or decontaminated at the corresponding contact surfaces or contact points.

In subsequent method step S4, the liquid decontamination agent may advantageously get from the containers via the closeable couplings, discharge points 09 and, thus, into gas line 07. The containers can be emptied either by hand, for example by actuating a piston, automatically, for example by designing the containers as cartridges, which, besides or in addition to the liquid decontamination agent, also have a propellant or propellant gas, or physically, for example by the effect of the carrier gas stream on the decontamination agent in the container in the manner of a jet nozzle or a Venturi nozzle. An introduction of the decontamination liquid or the liquid decontamination agent due to or with the aid of gravity/weight can also be advantageous.

After the containers have been emptied and the decontamination agent, in particular the liquid decontamination agent, has been conveyed by means of the carrier gas stream from discharge points 09 along gas lines 07 to atomizers 04 and, from there, into isolator space 02, the containers can be detached from the closable coupling in a further method step S5 and the closable coupling can be closed, if necessary after a repeated purification/decontamination.

A slightly modified method management using a decontamination assembly according to FIG. 1b is depicted in the illustration of FIG. 4. In the case at hand, in a first method step S1, used or utilized filters 11 can again be exchanged for new and preferably pre-sterilized or decontaminated filters 11. In a subsequent method step S2, decontamination agent, in particular liquid decontamination agent, is taken out of decontamination agent storage container 06 via connections 12 automatically or in a partially automated manner and provided at discharge points 09 of gas lines 07. In a subsequent method step S3, the liquid decontamination agent can be delivered from discharge points 09 through gas lines 07 and via atomizers 04 into isolator space 02, preferably as long as liquid decontamination agent is delivered in addition via a carrier gas stream by gas pressure source 08 in the direction of atomizers 04.

An alternative method management, preferably using a decontamination assembly according to FIG. 2 is depicted in FIG. 5. In the case at hand, a filter change as already described before can also advantageously be carried out in a first method step S1. In a method step S2, a desired concentration of decontamination agent in the form of decontamination aerosol can then first be produced with all atomizers 04 in isolator space 02 by guiding decontamination agent, for example hydrogen peroxide, to atomizers 04 via decontamination agent lines 05 and atomizing it by means of a carrier gas stream provided through gas lines 07 on the part of gas pressure source 08.

In a subsequent method step S3, individual atomizers 04 or groups of atomizers 04 may then take, in particular aspirate, decontamination aerosol from isolator space 02 and transport it through gas lines 07 in the direction of negative pressure source 13. For this purpose, valve 14 can be switched correspondingly such that, at least in individual or some gas lines 07, a corresponding negative pressure is generated and the connection to the carrier gas stream of gas pressure source 08 is interrupted. Meanwhile, other atomizers 04 may advantageously continue to generate decontamination aerosol by the supply of a carrier gas stream and decontamination agent and give it off into isolator space 02 in order to prevent the concentration of the decontamination agent in isolator space 02 from falling too quickly or too strongly. Valve 14 can be switched in such a manner for the last-mentioned group of atomizers 04 that the negative pressure of negative pressure source 13 cannot act on gas line 07 and that, instead, the carrier gas stream is applied to the atomizers and dispensed by them due to a positive pressure caused by the gas pressure source.

In a further method step S4 or in further method steps S4.1 to S4.N, further atomizers 04 can cause a sucking in of decontamination aerosol from isolator space 02 individually or in groups, while preferably other atomizers 04, as already described before, continue to give off decontamination aerosol into isolator space 02. For this purpose, valves 14 can be switched selectively.

In a subsequent method step S5, the residues of decontamination agent and/or decontamination aerosol possibly remaining in gas lines 07 can be emptied individually, in groups or simultaneously for respective gas lines 07 by switching valves 14 again and generating a carrier gas stream from gas pressure source 08 toward atomizers 04 in such a manner that residues can also be conveyed or blown out into isolator space 02 via gas lines 07 and atomizers 04.

### REFERENCE NUMERALS

01 decontamination assembly
02 isolator space
03 walls
04 atomizers
05 decontamination agent line
06 decontamination agent storage container
07 gas lines
08 gas pressure source
09 discharge points
10 closeable coupling
11 filter
12 connection
13 negative pressure source
14 valve
S1 method step
S2 method step
S3 method step
S4 method step
S5 method step

1. A decontamination method for applications in pharmaceutical engineering, comprising the steps of:
introducing a decontamination agent into at least one gas line (07) serving to supply at least one atomizer (04) with a carrier gas stream, said atomizer (04) being disposed in an isolator space (02), the at least one atomizer (04) being supplied with decontamination liquid from a decontamination agent storage container (06) via valve means controllable by means of electronic control means in order to generate a decontamination aerosol in a decontamination operation of the isolator space (02), the at least one atomizer (04) being connected to a gas pressure source (08) in a gas-conducting manner via the gas line (07) and a filter (11) for filtering the carrier gas stream being disposed in the gas line (07) between the gas pressure source (08) and the atomizers (04).

2. The decontamination method according to claim 1,
wherein
liquid decontamination agent is introduced into the gas line (07) and delivered with the carrier gas stream into the isolator space (02) via the atomizer (04).

3. The decontamination method according to claim 2,
wherein
the liquid decontamination agent is introduced at a discharge point (09) in the gas line (07), said discharge point (09) being individual for each atomizer (04).

4. The decontamination method according to claim 2,
wherein
the liquid decontamination agent is introduced at a discharge point (09) which is disposed in the gas line (07) between a filter (11) and the atomizer (04).

5. The decontamination method according to claim 2,
wherein
a container having a pre-dosed volume of liquid decontamination agent is coupled to and emptied at a closeable coupling (10) of the gas line (07) at the discharge point (09) during a temporary connection (12).

6. The decontamination method according to claim 2,
wherein
for decontaminating the gas line (07), the liquid decontamination agent is provided via a permanent feed line conveying decontamination agent from the decontamination liquid container (06) into the gas line (07) by means of corresponding conveying means, via an at least indirect connection (12) between the decontamination agent storage container (06) and the discharge point (09).

7. The decontamination method according to claim 1,
wherein
decontamination agent is introduced into the gas line (07) in the form of a decontamination aerosol.

8. The decontamination method according to claim 7,
wherein
the decontamination aerosol is introduced from the isolator space (02) into the gas line (07) and introduced into the gas line (07) by means of a carrier gas stream having a reverse flow direction to the carrier gas stream during the decontamination operation of the isolator space (02).

9. The decontamination method according to claim 7,
wherein
a first part of an entirety of several atomizers (04) atomizes a decontamination aerosol in the isolator space (02) while a second part of the entirety of the atomizers (04) takes the decontamination aerosol from the isolator space (02) and introduces it into the gas line (07) of the second part of the entirety of the atomizers (04).

10. The decontamination method according to claim 7,
wherein
the entirety of the atomizers (04) atomizes a decontamination aerosol into the isolator space (02) before introducing the decontamination aerosol into the gas line (07).

11. The decontamination method according to claim 7,
wherein
the flow direction of the carrier gas stream is reversed after the introduction of the decontamination aerosol into the gas line (07) and liquid, decontamination agent and/or decontamination aerosol remaining in the gas line (07) is delivered via the atomizers (04) into the isolator space (02) by means of the carrier gas stream.

12. A decontamination assembly (01) for applications in pharmaceutical engineering for performing the decontamination method according to claim 1, said decontamination assembly (01) comprising at least one isolator space (02) and
a decontamination agent storage container (06) from which at least one atomizers (04), which are disposed in the isolator space (02) and are realized as two-fluid nozzles, are suppliable with decontamination liquid via valve means controllable by means of electronic control means in order to generate a decontamination aerosol, the atomizers (04) being connected to a gas pressure source (08) in a gas-conducting manner via a gas line (07) in order to generate a carrier gas stream for a decontamination aerosol to be generated and a filter (11) for filtering the carrier gas stream being disposed in the gas line (07) between the gas pressure source (08) and the atomizers (04) individually for each atomizer (04),
wherein
the distance between the filter (11) and the atomizer (04) in a gas line (07) is less than 5 m and/or wherein the gas-conducting connection (12) comprises a discharge point (09) between the gas pressure source (08) and the atomizers (04), said discharge point (09) being provided individually for each atomizer (04) and/or filter

(11) and serving for the discharge of decontamination liquid.

13. The decontamination assembly according to claim 12,
wherein
at least one individual container for liquid decontamination agent is separably connected to a closeable coupling (10) provided at the discharge point (09).

14. The decontamination assembly according to claim 12,
wherein
the discharge point (09) is connected to the decontamination agent storage container (06) via an at least indirect connection (12).

15. The decontamination assembly according to claim 12,
wherein the decontamination assembly comprises a negative pressure source (13) which is connected in a fluid-conducting manner to a gas line (07) or a part of a gas line (07).

16. The decontamination method according to claim 1, wherein the atomizer (04) comprises a two-fluid nozzle.

17. The decontamination method according to claim 1, wherein the filter (11) is disposed in the gas line (07) individually for each atomizer (04) of the at least one atomizer (04).

18. The decontamination assembly according to claim 12, wherein the at least one atomizer (11) comprises several atomizers (04).

19. The decontamination assembly according to claim 15, wherein the negative pressure source is connected to the gas line (07) in a switchable manner.
